(19) United States
(12) Patent Application Publication
(10) Pub. No.: US 2008/0045934 A1
(43) Pub. Date: Feb. 21, 2008

(54) Device and method for coordinated insertion of a plurality of cryoprobes

(75) Inventors: Doris Schechter, Zikhron Yakov (IL); Gil Ofir, Even-Yehuda (IL); Ron Hillely, Zichron Yaakov (IL); Yaron Hefetz, Herzlia (IL); Mordechai Bliweis, Kiryat-Haim (IL)

Correspondence Address: Martin D. Moynihan;PRTSI, Inc., P.O. Box 16446, Arlington, VA, 22215, US

(73) Assignee: Galil Medical Ltd., Yokneam (IL)

(21) Appl. No.: 11/892,129

(22) Filed: Aug. 20, 2007

Related U.S. Application Data
(63) Continuation-in-part of application No. PCT/IL2007/000091, filed on Jan. 25, 2007.
Application No. PCT/IL2007/000091 is a continuation-in-part of application No. 11/637,095, filed on Dec. 12, 2006.
Application No. 11/637,095 is a continuation-in-part of application No. 11/660,478.
Application No. 11/660,478 is a continuation of application No. 09/860,486, filed on May 21, 2001, now Pat. No. 6,706,037.
Application No. 11/637,095 is a continuation-in-part of application No. 11/055,597, filed on Feb. 11, 2005.
Application No. 11/055,597 is a continuation of application No. 09/987,689, filed on Nov. 15, 2001, now abandoned.
Application No. 09/987,689 is a continuation of application No. 09/860,486, filed on May 21, 2001, now Pat. No. 6,706,037.
Application No. 11/637,095 is a continuation-in-part of application No. 11/185,699, filed on Jul. 21, 2005, now abandoned.
Application No. 11/185,699 is a division of application No. 10/151,310, filed on May 21, 2002, now abandoned.
Application No. PCT/IL2007/000091 is a continuation-in-part of application No. 11/640,309, filed on Dec. 18, 2006.
Application No. 11/640,309 is a continuation-in-part of application No. 10/660,478, filed on Sep. 12, 2003, now Pat. No. 7,150,743.
Application No. 10/660,478 is a continuation of application No. 09/860,486, filed on May 21, 2001, now Pat. No. 6,706,037.
(60) Provisional application No. 60/242,455, filed on Oct. 24, 2000.
(60) Provisional application No. 60/300,097, filed on Jun. 25, 2001.
(60) Provisional application No. 60/291,990, filed on May 21, 2001.
(60) Provisional application No. 60/762,110, filed on Jan. 26, 2006.
(60) Provisional application No. 60/750,833, filed on Dec. 16, 2005.

(51) Int. Cl. A61B 18/02

(52) U.S. Cl. 606/21

(57) ABSTRACT

The present invention relates to use of an introducer for delivering multiple thermal ablation probes to an organic target in a compact configuration, which probes are operable to be deployed in a dispersed configuration characterized by known and accurately maintained distances of one treatment head from another, which distances are substantially maintained during insertion of treatment heads into target tissues. In preferred embodiments, distal portions of deployed probe treatment heads are substantially parallel one to another.

### RELATED APPLICATIONS

This Application is a continuation-in-part of PCT Patent Application No. PCT/IL2007/000091 filed Jan. 25, 2007, which is a continuation-in-part of pending U.S. patent application Ser. No. 11/637,095 filed Dec. 12, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

U.S. patent application Ser. No. 11/637,095 is also a continuation-in-part of pending U.S. patent application Ser. No. 11/055,597 filed Feb. 11, 2005, which is a continuation of U.S. patent application Ser. No. 09/987,689 filed Nov. 15, 2001, now abandoned, which is a continuation-in-part of U.S. patent application Ser. No. 09/860,486 filed May 21, 2001, now U.S. Pat. No. 6,706,037, which claims the benefit of U.S. Provisional Patent Application No. 60/242,455, filed Oct. 24, 2000.

U.S. patent application Ser. No. 11/637,095 is also a continuation-in-part of U.S. patent application Ser. No. 11/185,699 filed Jul. 21, 2005, now abandoned, which is a divisional of U.S. patent application Ser. No. 10/151,310 filed May 21, 2002, now abandoned, which claims the benefit of U.S. Provisional Patent Application No. 60/300,097 filed Jun. 25, 2001, now expired, and U.S. Provisional Patent Application No. 60/291,990 filed May 21, 2001, now expired.

U.S. patent application Ser. No. 11/637,095 also claims the benefit of U.S. Provisional Patent Application No. 60/762,110 filed Jan. 26, 2006, now expired.

U.S. patent application Ser. No. 11/637,095 further claims the benefit of U.S. Provisional Patent Application No. 60/750,833 filed Dec. 16, 2005, now expired.

PCT Patent Application No. PCT/IL2007/000091 filed Jan. 25, 2007 is also a continuation-in-part of pending U.S. patent application Ser. No. 11/640,309 filed Dec. 18, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

This Application is also being filed concurrently with U.S. National Phase Patent Application No. ______ filed ______, titled “DEVICE AND METHOD FOR COORDINATED INSERTION OF A PLURALITY OF CRYOPROBES” (Attorney Docket No. 31852).

This Application is also being filed concurrently with U.S. continuation-in-part (CIP) Patent Application No. ______ filed ______, titled “THIN UNINSULATED CRYOPROBE AND INSULATING PROBE INTRODUCER” (Attorney Docket No. 37193).

This Application is also being filed concurrently with U.S. continuation-in-part (CIP) Patent Application No. ______ filed ______, titled “DEVICE AND METHOD FOR COORDINATED INSERTION OF A PLURALITY OF CRYOPROBES” (Attorney Docket No. 37225).

The contents of all the above-mentioned applications are incorporated herein by reference.

### FIELD AND BACKGROUND OF THE INVENTION

The present invention relates to devices and methods for thermal ablation of a surgical target within a body of a patient. More particularly, the present invention relates to use of an introducer for delivering thermal ablation probes to an organic target in a desired configuration and orientation.

Cryotherapy is often called upon to treat lesions which are larger than the size of the ice ball which can be formed by a single cryoprobe. Using, repositioning, and re-using a same probe to treat large lesion is impractical, given the time-consuming freezing, thawing, and re-freezing processes involved. Consequently, a plurality of probes is typically used to treat a large treatment target. Yet, it is difficult to accurately insert a plurality of cryoprobes into a body and to position those probes in such manner that their treatment heads are in desired target locations relative one to another and relative to a target lesion. The process is particularly difficult when long, thin cryoprobes are used. Yet treated organs are often deep within the body, and cryoprobes and associated sensor probes must penetrate thick layers of tissue to reach an intended treatment locus.

In prostate cryoablation, where insertion depth is relatively short, templates are used to guide a plurality of cryoprobes to a target. U.S. Pat. No. 6,142,991 to Schatzberger presents a system where a template facilitates cryoablation of a large lesion by directing a plurality of substantially parallel cryoprobes towards an ablation target. Schatzberger's template is positioned external to a patient's body. The template comprises a plurality of apertures, each aperture serving to guide movements of a single cryoprobe. U.S. Pat. No. 6,142,991 to Schatzberger is incorporated herein by reference.

Template systems similar to that of Schatzberger are appropriate only when the cryoablation target is so situated as to be accessible to cryoprobes directly penetrating through the skin from outside the body, as is the case in cryotreatment of the prostate, commonly accessed through the skin of the perineum. For ablation targets positioned deeper within the body, use of an external template to organize and direct a plurality of probes is impractical.

An alternative approach is presented by U.S. Pat. No. 6,706,037 to Zvuloni et al., and by PCT Application IL2007/000091 by Bliweis et al., both of which are incorporated herein by reference. These applications teach use of a sheath or “introducer” for introducing a plurality of cryoprobes into a body and delivering treatment heads of that plurality of cryoprobes to the vicinity of a cryoablation target, where that plurality of cryoprobe treatment heads may be used in concert to cryoablate or otherwise treat a designated target. Since such introducers are typically inserted into a body cavity through a trocar or an endoscope, it is important that the plurality of cryoprobes be contained and transported within the introducer in compact format. However, efficient cryoablation of a large target requires that the multiple probe treatment heads be inserted into the target in a dispersed or well-distributed format. PCT Application IL2007/000091 describes cryoprobe and introducer formats enabling cryoprobes to be introduced into a body in compact format and yet to be deployed in distributed format at a cryoablation target.

### SUMMARY OF THE INVENTION

Embodiments presented hereinbelow present apparatus and methods for use of an introducer for delivering thermal ablation probes to an organic target. In particular, embodiments are presented which may be used for delivering multiple probes in a configuration and orientation enabling efficient and thorough ablation of a large target of complex shape, wherein cryoprobe treatment heads are delivered to an ablation target vicinity in a compact configuration, yet are operable to be presented to that ablation target in a dispersed configuration characterized by known and accurately maintained distances of one treatment head from another, which distances are substantially maintained during insertion of treatment heads into target tissues. Preferred embodiments enable to deliver a plurality of treatment probes to a vicinity of a treatment target in a space-saving compact format, and there to expand the probes treatment tip positions into a dispersed format wherein distal portions of probe treatment heads are substantially parallel one to another.

Under prior art methods for introducing a plurality of cryoprobes into a body in compact configuration and expanding operating tips of those probes into a dispersed configuration within the body, probes introduced into a body through an introducer diverge from one another when distally advanced from within the introducer in a vicinity of an ablation target, and continue to diverge from each other in a continuous process as they continue to be so advanced. According to these devices and methods, the eventual distance of the plurality of cryoprobe treatment heads one from another is a function of the distance by which the probes are extended beyond the introducer and into their target. Treatment head positions, and in particular the distance between one head and another, are consequently somewhat difficult to predict and to control. Yet for many cryoablation tasks, and in particular for cryoablation of tumors, accurate control of distance of one treatment head from another is critical, because treatment heads too close together cause inefficiency in the treatment process, yet treatment heads too far apart risk leaving tumor cells, potentially capable of cancerous proliferation, insufficiently cooled to be fully and reliably destroyed by the cryoablation process.

Embodiments presented herein provide apparatus and methods enabling to deliver a compact configuration of a plurality of cryoprobe treatment tips to a vicinity of a treatment target deep within a body, and to there effect a dispersed distribution of treatment tips thus introduced, while maintaining a desired distance between and among those treatment tips during advancement of a plurality of cryoprobe treatment tips into a treatment target.

In presently preferred methods of use of some embodiments of the present invention, treatment probes are packed into an introducer, a distal portion of the introducer is caused to penetrate into a body cavity (either by use of a sharpened distal introducer edge or by use of a trocar), distal heads of the introduced treatment probes are caused to extend beyond a distal end of the introducer, and the extended heads are then caused to extend away from each other in a pre-planned configuration, preferably ending with their distal ends substantially parallel one to another. Forward pressure on probes and/or introducer is then applied, forcing sharp distal ends of the advanced and configured probe treatment heads to advance into an ablation target in a pre-planned configuration.

In some embodiments of the present invention the treatment heads, thus advanced, approximately maintain a constant distance one to another them as they advance in unison towards and into a target, so that distance between one treatment head and another is substantially independent of the depth to which the treatment heads are caused to penetrate into the target. This is in contrast to methods of prior art mentioned above, wherein probes extending from an introducer in dispersed configuration are necessarily angled outwards. In such prior art configurations, increasing depth of penetration into a target therefore implies increasing separation between treatment heads. Such prior art configurations limit the freedom of a surgeon in manipulating his probes. Further, such prior art configurations result in some difficulty in predicting or calculating distances between deployed probe heads and are therefore subject to errors in probe head placement, which errors may expose patients to inadequate ablation of probe heads are more distant from each other than planned or intended by the surgeon. Inadequate ablation of cancerous tissue can seriously endanger a patient. In contrast, a probe/introducer combination according to an embodiment of the present invention maintains a planned distance between probe treatment tips as those treatment tips are advanced into target tissue, thus providing advantages of convenience and safety, for a variety of clinical applications.

In a preferred method of use, the configuration of the distributed probe heads is pre-selected in consideration of known cooling characteristics of the probes, and is such as to facilitate efficient and thorough cryoablation of a target.

The present invention further successfully addresses the shortcomings of the presently known configurations by providing delivery of cryoprobe treatment heads to a target deep within the body in a distributed configuration wherein distal portions of said treatment heads are aimed and oriented in directions substantially parallel one to another, thereby enabling a plurality of treatment heads to be advanced in unison towards and into an ablation target in a well-controlled distributed configuration and at a pre-planned distance one from another.

Some embodiments of the present invention comprise an introducer containing pre-bent probes moveable therein, each having a pre-bent S-shaped curve, such that each probe tip, as it extends beyond a distal portion of the introducer, first curves away from a longitudinal axis of the introducer and then curves back so as to advance in a direction substantially parallel to said longitudinal axis of the introducer, but at an increased distance therefrom. Similar embodiments utilize probes comprising shape memory metal operable to assume a S-shaped curvature during the probe introduction process.

Additional embodiments comprise an introducer containing a plurality of probes with flexible distal portions, an inflatable balloon positioned at a distal portion of the introducer and between the probes, and a constraining attachment. In these embodiments probes and deflated balloon may be introduced into a body in compact configuration, then extended distally so that a distal portion of probes and at least a portion of the balloon extend beyond a distal end of the introducer, whereupon the balloon may be inflated, forcing distal ends of the plurality of treatment probes to separate one from another to a desired distance, which distance may be controllable dependant on degree of inflation of the balloon and/or may be limited by a constraining attachment serving to limit expansion of the probes away from the introducer and forcing distal ends of the treatment probes to aim in desired directions. In particular, balloons and constraining attachments can be used to force distal ends of treatment probes to aim in directions substantially parallel to each other and parallel to the longitudinal axis of the introducer.

In further embodiments particularly useful for treating lesions near the skin (i.e. near external portions of the body), a set of preferably parallel ablation needles extend from a common handle or common shaft.

In further embodiments, distal portions of a set of probes are attached to a common shaft by mechanical linkages, which linkages provide a first configuration which is highly compact and appropriate for the probes when contained in the introducer body during insertion into a body cavity, and a second configuration enabling dispersed deployment of a plurality of substantially parallel cryoprobe treatment heads, which deployment is appropriate for parallel insertion of the plurality of treatment heads into an ablation target. In some embodiments a parallelogram linkage is expanded away from the introducer's longitudinal axis when a cord or wire is pulled by an operator, causing a change in the internal angles of the parallelogram linkage. In alternative embodiments an X-shaped linkage is controlled by pushing and pulling a rod, causing a heightening or flattening of an X-shaped linkage, wherein flattening of the X-shaped linkage causes one or more cryoprobes to approach a central (i.e. longitudinal) axis, thereby inducing a compact configuration of probes appropriate for insertion within an introducer and for insertion into a body through that introducer, and wherein heightening of the X-shaped linkage causes distancing of a cryoprobe head from the central axis, thereby causing a set of probes so linked to assume a distributed parallel configuration with probe heads distanced to a controlled degree from a central axis, the heads being aimed and oriented so as to be substantially parallel to each other.

According to one aspect of the present invention there is provided a probe deployment apparatus comprising an introducer and a plurality of probes each having a probe head, the apparatus providing a compact configuration wherein the probe heads are contained within the introducer and a dispersed configuration wherein the probe heads extend beyond the introducer and distal tips of the probe heads are substantially parallel one to another.

According to further features in preferred embodiments of the invention described below, in the dispersed configuration, the distal tips are substantially parallel to a longitudinal axis of the introducer.

According to further features in preferred embodiments of the invention described below, at least one of the probes i comprises a cryocooler.

According to further features in preferred embodiments of the invention described below, the probe heads assume a compact configuration when constrained by being contained within the introducer, and assume a dispersed configuration when unconstrained.

According to further features in preferred embodiments of the invention described below, the probe heads are operable to be advanced and retracted within the introducer.

The probe heads may be constrained to advance and retract in unison, or alternatively at least one of the probe heads may be advanced and retracted independently of another of the probe heads.

According to further features in preferred embodiments of the invention described below, at least one of the probe heads comprises shape memory metal. In some embodiments at least one probe head comprises a cryocooler, and further comprises a portion which comprises shape memory metal which assumes an S-shaped configuration at a first temperature and another configuration at a second temperature.

According to further features in preferred embodiments of the invention described below, at least one of the probe heads is pre-bent and assumes an S-shaped curve when unconstrained.

According to further features in preferred embodiments of the invention described below, the plurality of probe heads extend from a common proximal shaft.

According to further features in preferred embodiments of the invention described below, at least two of the probe heads comprise cryocoolers and the common shaft comprises a common cryogen exhaust lumen communicating with the cryocoolers. In an alternate construction, at least two of the probe heads comprise cryocoolers and the common shaft contains a plurality of cryogen exhaust lumens each communicating with one of the cryocoolers.

Some embodiments comprise a plurality of independently moveable probes.

In some embodiments a plurality of the probes are cryoprobes each having a cryogen input lumen and a cryogen exhaust lumen. The apparatus may further comprise a binding which constrains at least some of the plurality of probes to advance and retract together.

According to further features in preferred embodiments of the invention described below, the probe heads are operable to assume a distributed configuration wider than a diameter of the introducer when extended beyond the introducer.

Preferably, a plurality of the probe heads comprise cryoprobe treatment tips coolable to cryoablation temperatures, and the treatment tips when in the dispersed configuration are operable to create a continuous cryoablation volume.

In some embodiments, while in the dispersed configuration, a first of the treatment tips is operable to create a cryoablation volume of radius D1 when operated in isolation, a second treatment tip, closest among the plurality of treatment tips to the first treatment tip, is operable to create a cryoablation volume of radius D2 when operated in isolation, and a distance D3 between the first treatment tip and the second treatment tip is greater than D1+D2.

Some embodiments further comprise an inflatable balloon, and the balloon is operable to enforce separation of distal portions of the probe heads when the inflatable balloon is inflated.

Optionally, the balloon may be annular in shape, having a central hole shaped to permit passage of a probe therethrough.

Alternatively, the apparatus may comprise a mechanical linkage which controls distances of the distal tips of the probe heads from a longitudinal axis of the introducer.

According to further features in preferred embodiments of the invention described below, the mechanical linkage comprises components formed as a parallelogram, and may comprise a spring tending to maintain the mechanical linkage in compact position or a spring tending to maintain the mechanical linkage in expanded position.

The apparatus may also comprise a pulling device which, when pulled, causes the mechanical linkage to assume an expanded configuration.

According to further features in preferred embodiments of the invention described below, the mechanical linkage comprises a plurality of components formed as an X-shaped construction having a central pivot. The mechanical linkage may further comprise a central rod which causes the linkage to expand laterally when pulled and causes the linkage to contract laterally when pushed.

According to a further aspect of the present invention there is provided a therapy apparatus which comprises a therapeutic probe having a base and a plurality of probe heads extending from the base in substantially parallel directions. Preferably, these probe heads are coolable. In some embodiments the plurality of the probe heads comprise cryocoolers, and the base comprises a common cryogen exhaust lumen operable to exhaust cryogen from the cryocoolers. Alternatively, the base may comprise a plurality of gas exhaust lumens each operable to exhaust cryogen from one of the cryocoolers.

According to further features in preferred embodiments of the invention described below, the apparatus further comprises a sheath having a distal face which comprises a plurality of guide apertures, each guide aperture sized to accommodate one of the probe heads, at least some of the probe heads being operable to advance through one of the guiding apertures when the base is advanced within the sheath. The sheath may comprise thermally insulating material.

According to a further aspect of the present invention there is provided a method for delivering therapeutic probes to a therapeutic target, comprising providing a probe deployment apparatus comprising an introducer and a plurality of probes each having a probe head, the apparatus providing a compact configuration wherein the probe heads are contained within the introducer and a dispersed configuration wherein the probe heads extend beyond the introducer and distal tips of the probe heads are substantially parallel one to another, positioning the probe heads within the introducer in the compact configuration, introducing a distal portion of the introducer into a body cavity, extending the probe heads from the introducer in the dispersed configuration, and advancing the dispersed probe heads towards and into a treatment target.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a multi-headed cryoprobe and associated introducer, according to an embodiment of the present invention;

FIG. 2 is a simplified schematic of cryoprobe/introducer combination comprising S-shaped pre-bent cryoprobes, according to an embodiment of the present invention;

FIG. 3 is a simplified schematic of the cryoprobe/introducer combination of FIG. 2, with pre-bent cryoprobe treatment heads extending distally beyond a distal end of the introducer, according to an embodiment of the present invention;

FIGS. 4a and 4b are simplified schematics showing alternative configurations of cryoprobes extending from the cryoprobe introducer of FIG. 3, according to embodiments of the present invention;

FIG. 5 is a simplified schematic showing exemplary dimensions of the cryoprobe/introducer combination of FIG. 3, and of the ablation zones produced thereby, according to an embodiment of the present invention;

FIGS. 6a is a simplified schematic showing relative dimensions of a compact (i.e. within the introducer) configuration of cryoprobes of FIG. 3, according to an embodiment of the present invention;

FIGS. 6b and 6c are simplified schematics showing relative dimensions of expanded (i.e. extended from the introducer) configurations of cryoprobes of FIG. 3, according to embodiments of the present invention;

FIGS. 7a and 7b are simplified schematics showing relative dimensions of compact and expanded configurations of the apparatus of FIG. 3, showing an embodiment comprising seven cryoprobes, according to an embodiment of the present invention;

FIG. 8a is a simplified schematic of an introducer/cryoprobe combination comprising a balloon for forcing extended cryoprobes into an expanded configuration, according to an embodiment of the present invention;

FIGS. 8b and 8c are simplified schematics respectively showing compact and expanded configurations of the cryoprobes of FIG. 8a, according to an embodiment of the present invention;

FIG. 9a is a simplified schematic of an introducer/cryoprobe combination comprising an annular balloon for forcing extended cryoprobes into an expanded configuration, according to an embodiment of the present invention;

FIG. 9b is a simplified schematic showing an expanded configuration of the cryoprobes and balloon of FIG. 9a, according to an embodiment of the present invention;

FIGS. 10a and 10b are simplified schematics of compact and extended configurations respectively of an introducer/probe combination utilizing a mechanical linkage for parallel extension of cryoprobes, according to an embodiment of the present invention;

FIG. 10c is a simplified schematic of the apparatus of FIGS. 10a and 10b, showing additional methods for control of deployment, according to an embodiment of the present invention; and

FIGS. 11a and 11b are simplified schematics of compact and extended configurations respectively of an alternative configuration of introducer/probe combination utilizing a mechanical linkage for parallel extension of cryoprobes, according to an embodiment of the present invention.

### DESCRIPTION OF THE PREFERRED EMBODIMENTS

The present invention relates to devices and methods for thermal ablation of a surgical target within a body of a patient. Specifically, the present invention can be used to introduce a plurality of cryoprobes in a compact configuration within an introducer into a body cavity, to extend operating tips of those probes from that introducer, to deploy operating tips of the probes in an expanded configuration broader in at least one dimension than the introducer within which the probes were introduced, distal ends of the probe tips being oriented substantially parallel to each other and being operable to approximately maintain a fixed distance one from another while being advanced towards and into an ablation target.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

As used herein, the term “cryocooler” refers to a Joule Thomson cooler, or a cooling device which cools by evaporation of a liquid cryogen, or any other type of device operable to cool to temperatures useable for cryotherapy.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperatures by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

For exemplary purposes, the present invention is principally described in the following with reference to an exemplary context, namely that of cryoablation of a treatment target by use of cryoprobes operable to cool tissues to cryoablation temperatures. It is to be understood that invention is not limited to that exemplary context. The invention is, in general, relevant to delivery of a plurality of therapeutic probes of any sort to a vicinity of an organic target in the described probe configurations, for any surgical use. Thus, though for simplicity of exposition, cryoprobes are presented in the Figures and reference is made to cryoprobes hereinbelow, yet all such references are to be understood to be exemplary and not limiting. Thus, discussion of cryoprobes hereinbelow are to be understood to apply also to therapeutic probes of other sorts. Similarly, references to cryoablation of tissues are also to be understood as exemplary and not limiting. Thus, references to cryoablation are to be understood as referring also to non-cryogenic thermal ablation, and to non-ablative cryogenic treatment of tissues. For example, in the discussion of probe delivery devices presented below, the probes described may be any treatment devices, such as RF antennas, or thermal heaters, or radioactive detachable treatment sources, or any similar therapeutic or diagnostic probes, as well as cooling cryoprobes. Thus, unless specifically related to a cooling context, references to “cryoprobes” herein are to be understood as referring to therapeutic probes in general, and to cryoprobes as a specific example thereof

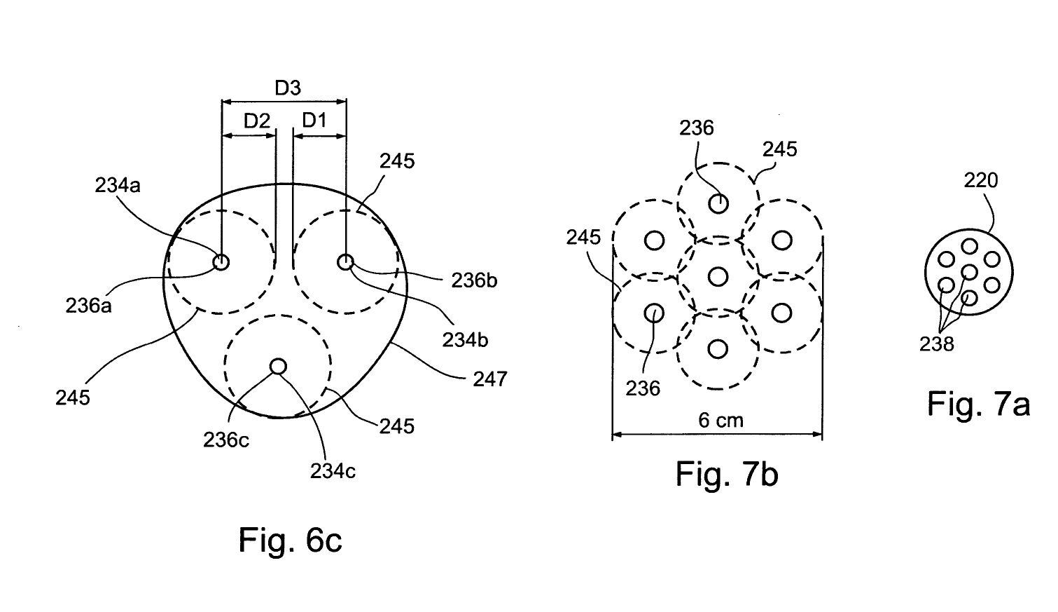

It is to be noted, however, that although useful in a variety of contexts, the !embodiments presented herein are of particular relevance and utility in the field of cryoablation. As will be discussed hereinbelow with particular reference to FIGS. 6b and 6c, the distances between inserted cryoprobe tips used in cryoablation can be of critical importance, particularly when ablating a malignant tumor. Embodiments of the present invention enable to control distance between probes in a manner which is independent of the depth to which the probes are inserted into a target, thereby providing an important advantage over prior art probe/introducer combinations.

It is expected that during the life of this patent many relevant cryoprobes and cryoprobe introducers will be developed, and the scope of the terms “cryoprobe” and “introducer” is intended to include all such new technologies a priori.

As used herein the terms “about” and “approximately” refer to ±20%, and references to objects being “substantially parallel” refer to objects which deviate from parallel orientations by angles of up to 20°, but preferably 10° or less.

In discussion of the various figures described hereinbelow, like numbers refer to like parts. The drawings are generally not to scale For clarity, non-essential elements are omitted from some of the drawings. In particular, to ensure clarity of the Figures, details of cryogen supply and exhaust systems within cryoprobes are not shown in the Figures, as such details are well known in the art and are not required for understanding of the invention presented herein.

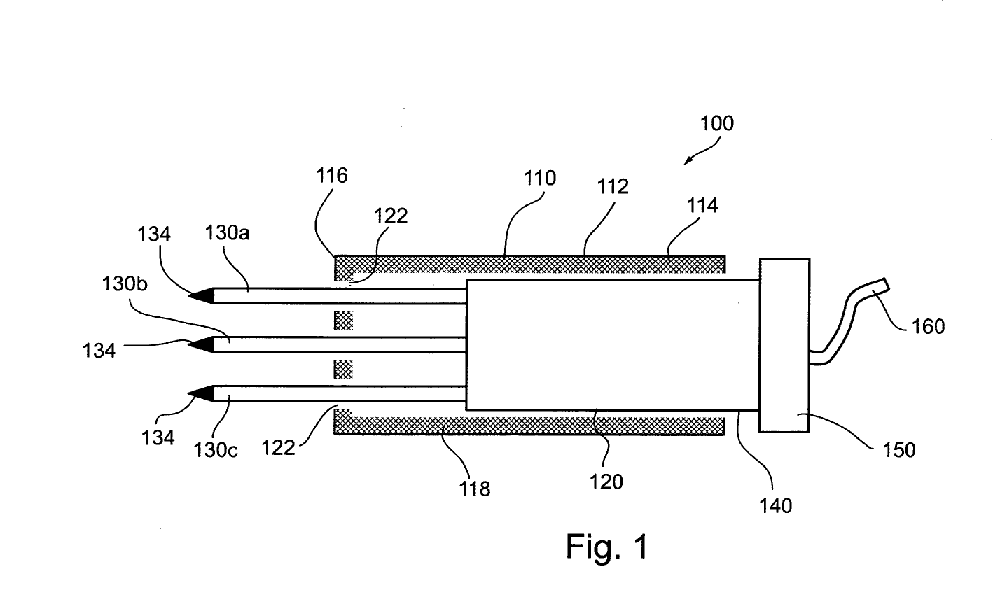

FIGS. 1-11, discussed hereinbelow, present probes and probe/introducer combinations useable in a variety of clinical contexts. It is noted, however, that a primary recommended use of the embodiments presented is in endoscopic surgery. For example, in a preferred method of used directed to surgery of the abdominal cavity, a trocar is inserted in a body wall, the body cavity is inflated with a fluid (gas or liquid depending on the imaging modality used), and an endoscope containing within a working channel a cryosurgery apparatus, as described hereinbelow, is introduced into the body cavity. Imaging modalities such as direct vision through an optical apparatus or camera (preferably introduced through a second trocar), inserted or external ultrasound probe, or x-ray fluoroscopy, are preferably used to guide placement of the endoscope distal portion with respect to a cryoablation target. Under guidance of a selected imaging modality, a surgeon introduces an endoscope/introducer through the trocar and into the cavity, advances the endoscope towards a selected ablation target, extends cryoprobe treatment heads beyond walls of the endoscope (typically, beyond a distal end of the endoscope), and, having aimed and aligned cryoprobe treatment heads with his intended target, further advances treatment heads and/or endoscope so as to cause the treatment heads to penetrate the ablation target. Treatment heads are then activated in cooling and subsequently optionally also activated in heating, according to well-know clinical treatment protocols, thereby ablating the target. After cooling and then optional heating to enable disengagement, probe treatment heads are then retracted into the endoscope and the endoscope retracted from the body.

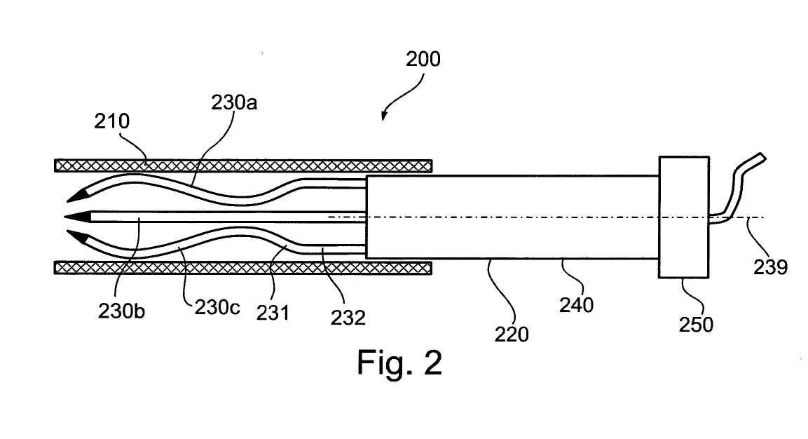

With respect to embodiments described by FIGS. 2-11 and similar embodiments, according to a recommended method of use, subsequent to introduction of the endoscope into the body cavity and prior to insertion of treatment heads into a target, treatment heads initially contained within the endoscope in a compact configuration are extended from the endoscope in an expanded configuration wherein treatment heads are positioned parallel one to another, and at a distance one from another which is greater (in at least one dimension) than the distance by which they are separated when in compact configuration. According to this recommended method of us, deployment of treatment heads into expanded configuration takes place within the body cavity and prior to insertion of the treatment heads into target tissues. In preferred embodiments, cryoprobe heads thus deployed are operable to be inserted in parallel into an ablation target, at a distance one from another which, in at least one dimension, is greater than the diameter of the endoscope within which they are introduced, and which ensures a desirable cooling pattern for ablation purposes. Details of these procedures are discussed more fully hereinbelow, with respect to various embodiments utilizing a variety of methods of treatment head deployment.

Attention is now drawn to FIG. 1, which is a simplified schematic of a multi-headed cryoprobe and associated introducer, according to an embodiment of the present invention. FIG. 1 presents an apparatus 100 which comprises an optional sheath 110 and a multi-headed cryoprobe 120. Multi-headed cryoprobe 120 comprises a common shaft 140 and a plurality of cryoprobe heads 130. Three cryoprobe heads here labeled 130a, 130b and 130c are presented in FIG. 1, yet that number of heads is exemplary and not limiting: more or fewer heads may be presented. Probe 120 is characterized in that probe heads 130, each comprising a treatment head 134 which comprises a cryocooler, are oriented so as to be substantially parallel to each other.

The U.S. patent application entitled “Device and Method for Coordinated Insertion of a Plurality of Cryoprobes” cited hereinabove and which is incorporated herein by reference, teaches a variety of configurations of multi-headed probes having a common shaft and therein a common cryogen exhaust lumen. Cryoprobe 120 of FIG. 1 may be any of the multi-headed probe configurations there presented, or indeed any other configuration of cryoprobe having a plurality of cooling heads and a common shaft or handle. In particular, probe 120 may have multiple cooling heads and a common cryogen exhaust lumen, such that cryogen used to cool individual cryoprobe heads exhausts from those heads into a common fluid manifold and is thence exhausted from the probe shaft in a common cryogen exhaust lumen, as taught therein.

Alternatively, the object referred to herein as “probe 120” may be constructed as a collection of individual probes (i.e. conventional prior-art probes each having an individual gas exhaust lumen). Individual probes comprising what is referred to herein as “probe 120” may be unconnected to each other and operable to be advanced and retracted individually, yet in a preferred embodiment such probes are bound together so as to be operable to be physically manipulated collectively (e.g. by grasping a shaft 140 containing shafts of individual probes). In a further alternative construction individual probes may be bound in sub-groups, which subgroups are operable to be advanced and retracted together. However, as shown in exemplary FIG. 1, probe 120 is either a group of individual probes so connected as to be operable to be manipulated (e.g. advanced and retracted) as a group, or a multi-headed probe comprising a plurality of treatment heads and a common shaft.

As stated above, probe 120 is characterized in that probe heads 130, each comprising a treatment head 134 which comprises a cryocooler, are oriented so as to be substantially parallel to each other. In consequence, a user may grasp probe 120 at shaft 140 or handle 150, and, by advancing probe 120 towards an ablation target, advance a plurality of probe heads 130 in unison towards and into that target, thereby bringing, with a simple motion of probe 120, a plurality of probes into positions appropriate for cryoablation of a large ablation target. Thus, in a preferred embodiment described above, probe 120 enables to ablate, with great simplicity, a target too large to be effectively ablated by a single probe.

Probe heads 130 are preferably parallel to each other, thus enabling simultaneous penetration of a target when the body of probe 120 is advanced toward that target. Preferably, probe heads 130 are separated one from another by a distance selected in view of a designed clinical purpose (e.g. prostate BPH ablation) and in view of known cooling characteristics of probe heads 130. In general, a cryoprobe used in a particular type of context (e.g. a prostate BPH ablation) and with a particular cryogen source (e.g. argon gas supplied to a probe 120 at a particular pressure), and used for a selected length of time, will ablate tissue within a known radius, thus creating a predictable ablation volume. Probe heads 130 are preferably positioned on probe 120 at distances which will create a continuous ablation volume, continuously incorporating individual ablation volumes created by individual probe heads 130, enabling to ablate tissues between heads 130 as well as tissues immediately contiguous to individual heads 130. Thus, probe 120 may be used with great simplicity, since the parallel configuration of probe heads 120 will in many clinical contexts guarantee that the distances between heads 130 will be at least approximately preserved when heads 130 are inserted into a target, and consequently that when a cryogen is supplied to probe 120 through a cryogen supply hose 160, causing cooling of treatment heads 134, a continuous ablation volume will result. Examples of continuous ablation volumes created in similar manner are presented in FIGS. 6 and 7 and discussed in detail hereinbelow.

An optional sheath 110 is provided for use with probe 120. Sheath 110 may be an introducer 112 designed for penetration into a body, or simply an external guide 114 designed to hold and to guide probe 120 while shaft 140 of probe 120 remains outside a body. If sheath 110 is designed as an introducer 112, it may be provided with either sharp or blunt distal edges 116, and may comprise thermally insulating material 118.

Sheath 110 is characterized by the presence of guide apertures 122, preferably positioned in a distal face of sheath 110, as shown in the Figure. Guide apertures 122 serve to guide probe heads 130 as probe 120 is advanced within sheath 110, and thus help to maintain correct direction and parallel orientation of probe heads 130 during advancement of probe 120 towards an organic target.

In a preferred embodiment of apparatus 100, probe heads 130 are constructed without insulation and may also be constructed without included heat exchangers, for example, as taught in the U.S. patent application “Thin Uninsulated Cryoprobe and Insulating Probe Introducer” cited hereinabove and which is incorporated herein by reference. Such configurations enable to construct extremely thin probe heads 130 which are advantageous in providing relatively easy penetration of probe heads 130 into target tissue and minimal peripheral damage to healthy tissue along the insertion path of heads 130. Guide apertures 122 are particularly useful for guiding and directing thin and flexible probes.

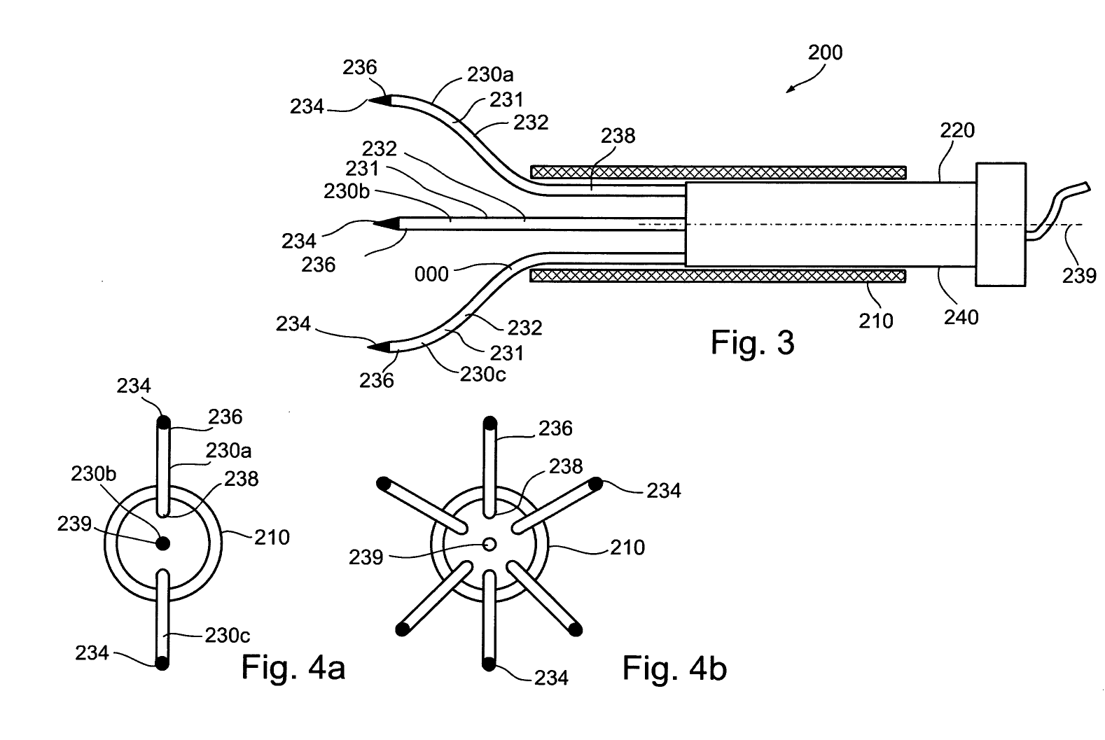

Attention is now drawn to FIGS. 2 and 3, which are simplified schematics of two views of a cryoprobe/introducer combination comprising S-shaped cryoprobe heads, according to an embodiment of the present invention.

FIGS. 2 and 3 present an apparatus 200 comprising a cryoprobe 220 shown -inserted in an introducer 210. Cryoprobe 220 comprises a common shaft 240, optional handle 250, and a plurality of cryoprobe heads 230. In similarity to apparatus 100 discussed hereinabove, shaft 240 of cryoprobe 220 may comprise a common cryogen exhaust lumen serving to exhaust cryogen from the plurality of cryoprobe heads 230, or alternatively the object referred to herein as “cryoprobe 220” may be constructed of a plurality of individual cryoprobes each having an individual distal cryoprobe head and an independent proximal shaft, which shafts are bound together to form an object referred to herein as “shaft 240” by means of which that plurality of individual cryoprobe shafts may be grasped and manipulated collectively (e.g. by grasping a “shaft 240” comprising a plurality of shafts of individual probes glued or otherwise attached to each other).

Three cryoprobe heads 230, here labeled 230a, 230b and 230c, are presented in FIGS. 2 and 3, yet that number of heads is exemplary and not limiting: more or fewer heads may be presented. Cryoprobe heads 230 comprise treatment tips 234, each of which comprises a cryocooler such as a Joule-Thomson cooler or an evaporative cooler cooled by evaporation of a liquid cryogen.

Probe 220 is characterized in that at least some of probe heads 230 are “pre-shaped” probe heads, meaning that the probe heads are manufactured to have a specific non-straight shape under certain conditions. In a preferred embodiment of apparatus 200 discussed in detail hereinbelow, heads 230 comprise an S-shaped curve under certain conditions. Two embodiments are presented in particular, one embodiment in which some or all heads 230 are “pre-bent” probe heads 231, manufactured to assume an S-shaped curve when not constrained to assume another configuration, and a second embodiment in which some or all heads 230 are shape memory metal probes 232 comprising a shape memory metal (e.g. Nitinol) manufactured to assume an S-shaped form at certain temperatures.

It is noted that the nature and use of pre-bent cryoprobes is presented in detail in PCT Application IL2007/000091, cited hereinabove and which is incorporated herein by reference. Pre-bent probes (e.g. comprising stainless steel) may be manufactured to be sufficiently flexible to be operable to fit within a straight and narrow introducer such as introducer 210 when constrained to do so, and to spring back to a pre-determined (“pre-bent”) shape when freed from constraints imposed by their containment within introducer 210. Thus, in a preferred embodiment some probe heads 230 are pre-bent probe heads 231 operable to be fitted within introducer 210 as shown in FIG. 2, and further operable to-assume an S-shaped configuration when advanced from within introducer 210 to a position where they are free of constraints imposed by introducer 210 and are free to assume their pre-bent shape, as shown in FIG. 3.

A preferred shape for heads 231 when unconstrained is shown in FIG. 3, which presents probe heads 230/231/232 advanced to a position within introducer 210 where a distal portion of their length is unconstrained by introducer 210. Probe heads 230a and 230c are there seen to assume an S-shaped curve, characterized in that a distal portion 236 of heads 230a and 230c and a proximal portion 238 of heads 230a and 230c may be seen to be oriented in substantially a same direction, which direction is preferably substantially parallel to a longitudinal axis 239 of introducer 210. On at least one and preferably most of probes 230, distal portions 236 may be seen to be laterally displaced with respect to a longitudinal axis 239 of introducer 210, as compared to corresponding proximal portions 238. This lateral displacement may be clearly seen in FIGS. 4a and 4b, which are simplified schematics showing end-on views of apparatus 200, according to embodiments of the present invention. FIG. 4a presents an embodiment where three probe heads 230 are present, two of which (230a and 230c) are S-shaped probes. FIG. 4b presents an alternative embodiment where six probe heads 230 are present, wherein a seventh centrally-located seventh probe is optional. It is of course to be understood that these specific numbers of heads are exemplary and not intended to be limiting.

It is to be noted that in a preferred embodiment presented in FIGS. 2, 3, and 4a, an un-bent (i.e. straight) central probe or probe head is provided, surrounded by S-shaped pre-bent probes or probe heads. This preferred configuration is particularly convenient in a variety of clinical situations. Preferably, the central probe head (230b in these Figures) is operable to be advanced independently of the other probe heads. In a preferred mode of use, a central probe is first advanced into a central portion of an ablation target, and fixed (i.e. attached) to that target at that position. A central probe may be attached to a target by being cooled to freezing temperatures, for example, thereby causing target tissues to adhere to the central probe. Alternatively, a central probe may comprise a grasping element. Further alternatively, a central probe may be formed with a hooking arrangement. In a currently preferred embodiment, central probe head 230b is formed in a corkscrew shape, and is rotated when advanced, thereby causing it to penetrate spirally into a target, thereby fixing it to that target.

In a preferred method of use, such a central probe is fixedly attached to a target using a hook or corkscrew or similar arrangement, and so stabilizing the spatial relationship of apparatus 200 to that target. Then, the plurality of probes or probe-heads surrounding the central probe may conveniently and accurately be advanced into the target.

As may be seen clearly in FIGS. 3, 4a and 4b, pre-shaped curves of probe heads 230 cause distal portions 236 of heads 230 to undergo lateral displacement (i.e. they become more distant from introducer longitudinal axis 239) when heads 230 are advanced from within into introducer 210 to an unconstrained position. As a result, a plurality of distal portions 236 of probe heads 230 expand to form an expanded configuration of distal portions 236 when advanced from introducer 210. As stated above, distal portions 236 comprise treatment tips 234, which comprise cryocoolers. For simplicity of exhibition, FIGS. 2-4 do not show internal details of cryocoolers L comprised in tips 234, no do they show details of internal probe features designed to supply and exhausted cryogen used for cooling tips 234, as these mechanisms are well known in the art. It is to be understood that treatment tips 234 may comprise cryocoolers such as Joule-Thomson coolers or evaporative coolers, and be operable to be positioned adjacent to or to be inserted into a cryoablation target and there to be cooled to cryoablation temperatures. Treatment tips 234 may also comprise sensor tips, such as thermal sensors, which may be combined with cooling tips so that a given tip both cools and registers temperatures, or may alternatively interspersed with cooling tips so that some tips cool, and other tips are provided to measure and report the cooling achieved.

A preferred method of use of apparatus 200 may be understood from FIGS. 2-4. FIG. 2 shows probe heads 230 attached to a common shaft 240 and handle 250 and operable to be advanced within introducer 210. When positioned as shown in FIG. 2, with probe heads 230 entirely contained within introducer 210 and positionally constrained thereby, heads 230 are in a compact configuration having a maximum diameter inferior to the diameter of introducer 210. FIG. 3 shows that probe 220 may be advanced within introducer 210 to a position whereat at least distal portions of heads 230 are unconstrained by introducer 210, whereupon these distal portions assume their unconstrained “pre-bent” shape as shown in FIG. 3, resulting in distal portions 236, and with them treatment tips 234, assuming an expanded configuration typically having a diameter significantly greater than the diameter of introducer 210. Thus, treatment tips 234 may be introduced to a vicinity of an ablation target in a compact configuration within a relatively narrow introducer (as shown in FIG. 2), may then be caused (by being advanced within introducer 210) to assume a relatively wide expanded configuration as shown in FIG. 3, and may then be further advanced in their expanded configuration towards and optionally into an ablation target, and there be used to treat a target lesion.

It is to be noted that, in a preferred embodiment, when probe heads 230 are in expanded configuration as shown in FIG. 3, distal probe head portions 236 are further distinguish by being oriented in a directional orientation which is substantially parallel to their proximal portions 238 and to longitudinal axis 239 of introducer 210. In a preferred embodiment, the S-shaped curves of heads 230 are so oriented as to cause lateral displacement of distal portions 236 away from central axis 239. Thus, distal portions 236 when in expanded configuration as shown in FIG. 3 are positioned at a distance one from another which is substantially greater than their distance one from another when positioned within introducer 210 in their compact state, the overall collective diameter of distal portions 236 is greater than the diameter of introducer 210, and distal portions 236 when in their extended configuration are substantially parallel to one another and to axis 239. Distal portions 236 are also preferably sharp and thus are shaped appropriately for penetrating an organic target.

In a preferred mode of use, introducer 210 containing probe 220 as shown in FIG. 2 is used to advance probe 220 into a body cavity, distal portions 236 are then sufficiently advanced from within introducer 210 to enable distal portions 236 to assume an expanded configuration as shown in FIG. 3, and then probe 220 is further advanced so that parallel distal head portions 236, comprising treatment tips 234, advance and penetrate a cryoablation target while substantially preserving the expanded configuration shown in FIG. 3. Apparatus 200 can thus be used to introduce a plurality of treatment tips in a predetermined configuration and at a predetermined distance one from another into a cryoablation target, wherein treatment tips 234 can be cooled to cryoablation temperatures to ablate the target. A plunger or control rod (not shown in the Figure) may be provided to facilitate controlled advancing of distal portions 236 from within introducer 210.

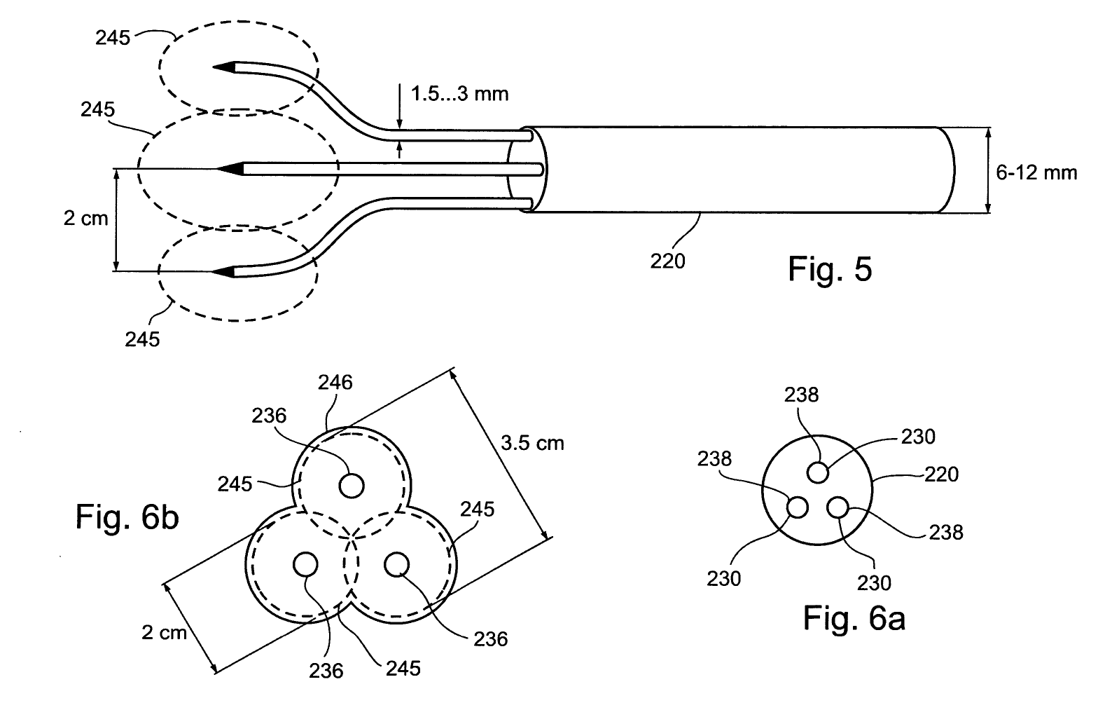

Attention is now drawn to FIG. 5, which shows dimensions of an exemplary and currently preferred embodiment of probe 220. Probe 220 is shown to have a diameter of between 6mm and 12mm, probe heads 230 are shown to have a diameter of between 1.5mm and 3mm, distal probe portions 236 are shown to be distanced one from another by approximately 2 cm when in expanded configuration. Ablation zones 245, zones of assured tissue destruction from cryoablation, are shown surrounding individual treatment tips 234 of distal portions 236 of probe heads 230.

Attention is now drawn to FIGS. 6a, 6b, and 6c, which are a simplified schematics of proximal (FIG. 6a) and distal (FIGS. 6b and 6b) portions of probe heads 230 in an exemplary probe 220 having three probe heads 230, according to an embodiment of the present invention. FIG. 6a shows proximal portions 238 of heads 230, where they attach to shaft 240 of probe 220. FIGS. 6b and 6c show distal portions 236 of those same heads 230, in expanded configuration. Hypothetical ablation zones labeled 245 show ablation volumes such as would be produced if each individual treatment tips 234 within distal portions 236 were operated in cryoablation individually (e.g. one at a time).

As shown in FIG. 6b, individual ablation zones 245 overlap, thereby creating a continuous collective ablation zone 246. The configuration shown in FIG. 6b might be termed a “conservative” configuration, reflecting a conservative approach to design of apparatus 200 (and similarly to the other probe deployment apparatus described hereinbelow). According to this “conservative” approach to design of apparatus 200 and other apparatus described below, positions of distal portions 236 when in deployed (expanded) configuration are calculated and manufactured to be such that individual ablation zones 245 (i.e. ablation zones which would be created by operation of treatment tips 234 operated individually) overlap slightly. Such a configuration guarantees production of an over-all ablation zone 246 as shown in FIG. 6b. When treating cancerous growth, some surgeons may prefer embodiments of apparatus 200 (and the other apparatus described according to principles taught herein) configured according to this conservative design.

Conservative design may be contrasted to an alternative design strategy presented by FIG. 6c. The configuration shown by FIG. 6c takes into account synergies presented by a plurality of cryoprobe treatment heads working in concert. Heat-transmission theory and simulations and experimental and clinical temperature measurements confirm that when heat is drawn from tissue at a plurality of neighboring points, ablation temperatures are reached in a volume 247 which exceeds the dimensions of the individual ablation volumes 245 which would be created by each probe tip, if each probe tip were used in isolation.

Therefore, in a presently preferred and recommended configuration presented in FIG. 6c and referred to herein as an “optimized” configuration, distal portions 236 are deployed to positions such that a distance D3 of one distal portion 236 from another is greater than then sum of the radii of their expected individual ablation zones 245. Thus, referring to FIG. 6c, if distance D2 is an expected radius of an ablation volume which would be created by treatment tip 234a operated in isolation, and distance D2 is an expected radius of an ablation volume which would be created by treatment tip 234b operated in isolation, then in a preferred embodiment of apparatus 200 (and of other apparatus presented hereinbelow), distance D3 separating treatment tip 234a from treatment tip 234b is greater than the sum of distance D1 plus distance D2. For example, for an apparatus 200 having treatment tips 234 individually capable of creating ablation volumes with a 1 cm radii (D1, D2) under a particular treatment protocol and in a particular type of tissue, optimal design of apparatus 200 might be to deploy such treatment heads at a distance D3 of 2.5 cm from each other. These dimensions are of course exemplary and not limiting. Exact determination of an optimal distance D3 is a function of individual characteristics of treatment tips 234a, 234b and 234c, of the treatment protocol selected for use (which protocol determines time and intensity of cooling, presence of proximate heat sources, etc.) and of the specific nature and in particular the specific heat transfer characteristics of tissues to be treated. Indeed, in a presently preferred method of design of apparatus 200 (and others presented herein), distances D3 between one deployed treatment head and another are first approximately determined according to theories of heat transfer and simulation methods well known in the art, and then verified and further optimized by experimental and/or clinical measurement.

It is to be noted that the “optimized” configuration presented by FIG. 6c #presents important advantages over the “conservative” configuration presented by FIG. 6b : the FIG. 6c design enables optimal use of cryoprobe cooling capacity, in that a significantly larger tissue volume can be cryoablated by a same set of cryoprobe treatment heads, as compared to that set of heads deployed in conservative configuration as presented by FIG. 6b. It is further to be noted that “optimized” cryoprobe treatment head deployment configurations, here presented in the exemplary context of apparatus 200, is equally useful and desirable in configuring other apparatus presented hereinbelow, and also in configuring other apparatus and devices for delivering a plurality of cryoprobes to an ablation target. In particular, an “optimized” configuration of probes can be used with advantage whenever an introducer is used to deliver a plurality of probes (endoscopically or otherwise) to an ablation target. Similarly, the optimized configuration can be used with advantage in designing and configuring multi-headed cryoprobes having multiple treatment heads connected to a common cryogen-transporting shaft.

Attention is now drawn to FIG. 7, which is a simplified schematic of proximal (FIG. 7a) and distal (FIG. 7b) portions of probe heads 230 in an exemplary probe 220 having seven probe heads 230, according to an embodiment of the present invention. FIGS. 7a and 7b are similar to FIGS. 6a and 6b in all respects, excepting, of course, a different number of probe heads. FIG. 7b, like FIG. 6a, shows that a continuous ablation volume may be created in a broad distributed configuration by cooling treatment tips in an expanded configuration of appropriately distanced operating tips positioned within an ablation target. FIG. 7b shows cryoprobe heads configured according to the “conservative” distribution discussed above, yet in many clinical contexts and alternative “optimized” configuration may be preferable, as discussed above.

It is to be noted that FIGS. 2-7 also represent an embodiment mentioned above, in which probe heads 230 are shape memory metal probe heads 232. In this alternative preferred embodiment, probe heads 232 are manufactured to assume an S-shaped configuration (as shown in FIG. 3) when cooled to below a selected temperature (e.g. when cooled to below −5° C.) Probe heads so configured may be positioned within introducer 210 in a collapsed configuration, extended from introducer 210 within a body cavity, then cooled to below that selected temperature, causing at least one and preferably most or all probe heads 232 to assume an S-shaped configuration as shown in FIG. 3, advanced into a target and used in cryoablation treatment as described hereinabove with reference to heads 231, then heated slightly to permit disengagement of heads 232 from frozen tissue and to enable heads 232 to resume their collapsed configuration, whereupon probe heads 232 may be retracted into introducer 210 and removed from the body.

It is further noted that whereas FIGS. 2-7 and the discussion hereinabove have represented probe heads 230 as being connected to a common shaft 240 and to be operable to be collectively manipulated (i.e. advanced and retracted within introducer 210) by manipulation of shaft 240 or optional handle 250, yet in a preferred alternative design and construction probe heads 230 may be connected to independent shafts 240a, 240b etc. (not shown), so as to be operable to be advanced and retracted individually. In a further alternative construction, a plurality of heads 230 may be connected to a restricted number of individual shafts 240a, 240b, etc., enabling sub-groups of probe heads 230 to be advanced and retracted in as sub-groups.

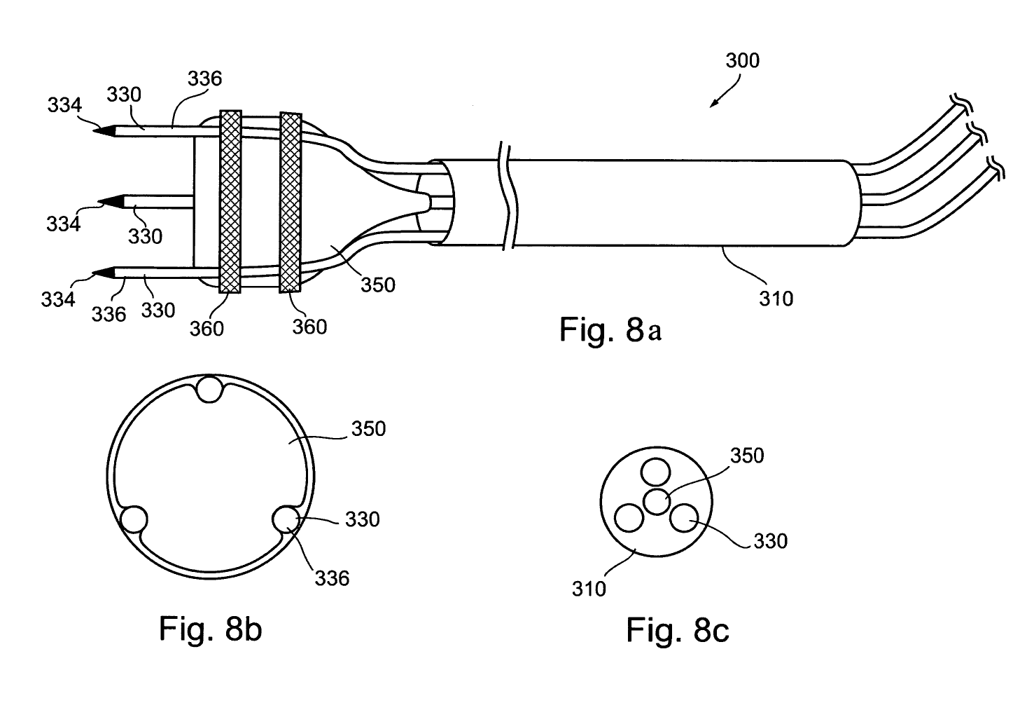

Attention is now drawn to FIGS. 8a, 8b, and 8c, which are simplified schematics of views of an apparatus 300 comprising an introducer 310 and a set of probe heads 330 (three are shown in this exemplary Figure) each comprising a cryosurgery treatment tip 334, according to an embodiment of the present invention.

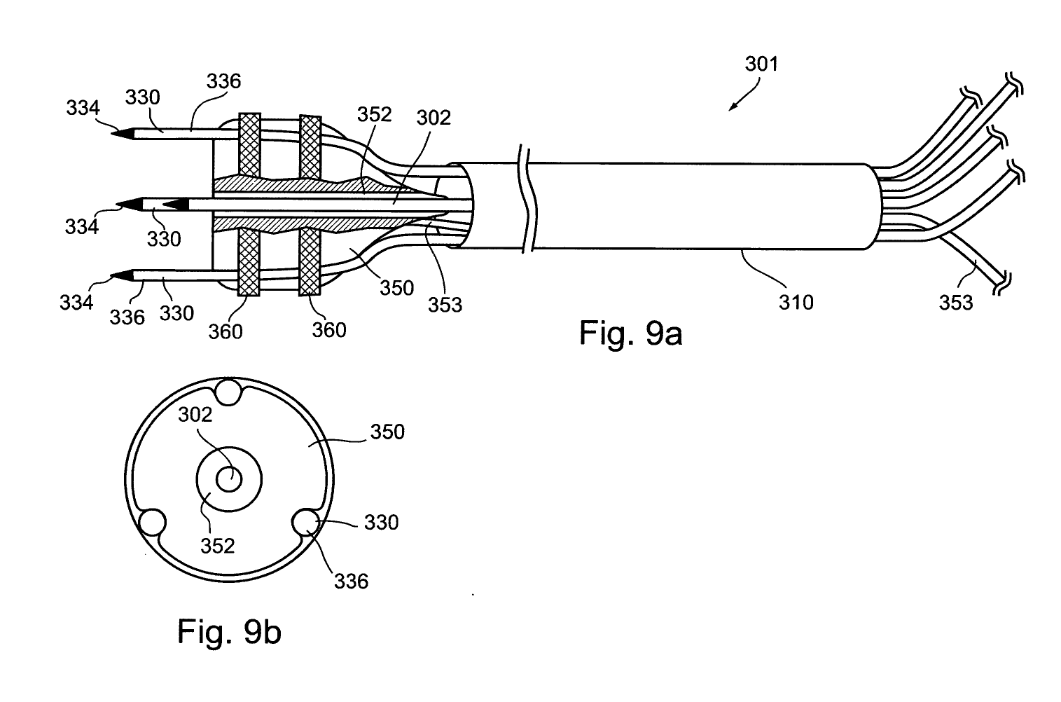

The general structure and use of apparatus 300 is in most respects similar to that of apparatus 200 described hereinabove. As seen in FIG. 8a, apparatus 300 differs from apparatus 200 in that apparatus 300 further comprises an inflatable balloon 350 and optional constriction band or plurality of bands 360. During use, balloon 350 is initially uninflated, and balloon 350 and probe heads 330 are contained within introducer 310, which may be used to introduce probe heads and balloon into a body cavity substantially as described hereinabove with respect to apparatus 200. Once introduced into a body cavity, distal portions of heads 330, together with all or part of balloon 350, may be caused to advance from introducer 310, in similarity to methods described above with reference to use of apparatus 200. Then, once distal portions of probe heads 330 and balloon 350 have been advanced beyond a distal end of introducer 310, balloon 350 may be inflated by fluid provided through an inflation/deplation tube (not shown in this Figure, see FIG. 9a), forcing apart distal portions 336 of probe heads 330 and causing distal portions 336 (and treatment tips 334 contained therein) to assume an expanded configuration. Distal portions 336 preferably extend for a distal distance of at least 4 cm beyond balloon 350, so that an iceball created by operation of treatment tips 334 will not extend to balloon 350. Optional constriction bands 360 are provided to limit expansion of balloon 350 and to assist in accurately controlling separation and positioning of distal portions 336 when balloon 350 is expanded. Alternatively or additionally, balloon 350 may comprise flexible yet unstretchable material such as cloth. Such an embodiment of balloon 350 would be inflatable only up to a pre-determined size and/or shape, thus combining the functionality of a continuously expandable balloon 350 together with the size and shape-restricting effect of constriction bands 360.

Once distal portions 336 are forced into expanded configuration by inflation of balloon 350, apparatus 300 may be used as described above with respect to apparatus 200. In a preferred configuration, balloon 350 and optional constriction bands 360 are sized and shaped to constrain distal portions 336 to be aligned in parallel to each other and to a longitudinal axis of apparatus 300 when balloon 350 is inflated. Distal portions of probe heads 330 are sufficiently rigid to resist buckling when inserted into tissue, yet comprises flexible or simi-rigid portions so as to be able to adapt appropriately to expansion of balloon 350. Embodiments may be prepared with a degree of rigidity/flexibility appropriate for specific tissue types or for specific clinical contexts.

FIGS. 8b and 8c provide additional views of apparatus 300, enabling to contrast the compact configuration of heads 330 within introducer 310 (FIG. 8b) with the expanded configuration of heads 330 (FIG. 8c) after balloon 350 has been extended from introducer 310 and inflated.

Attention is now drawn to FIGS. 9a and 9b, which are simplified schematics of an apparatus 301 comprising cryoprobe treatment heads, an introducer, and an expansion balloon. Apparatus 300 is similar to apparatus 300 described above, and differs therefrom in that a centrally positioned treatment probe 302 is provided, in characteristics and use similar to straight central probe 230b presented in FIGS. 2, 3, and 4 and discussed in detail hereinabove. Probe 302, in similarity to probe 230b, can be used to anchor apparatus 301 to a target tissue prior to deployment and/or insertion of probe heads 330. In similarity to probe 230b, probe 302 may itself be a cryoprobe with treatment head and cooling capabilities, or may be a simplified probe with anchoring functions but without cooling ability. Apparatus 301 comprises a balloon 351 similar in use and characteristics to balloon 350 of FIG. 8a, and differing therefrom in that balloon 351 has an annular shape, with a central channel or hole 352 through which probe 302 passes and is free to advance and retract. As may be seen FIGS. 9a and 9b, a plurality of probe heads 330 may be organized around a central probe 302. Central probe 302 may be used, as described with respect to probe-head 230b, to provide mechanical support during insertion into a target of probe heads 330 after those heads are deployed in expanded configuration by inflation of annular balloon 351. Balloon 351 is inflatable and deflatable through balloon inflation/deflation tube 353.

It is noted that channels such as channel 352 may be provided at other positions within balloon 351, and serve to guide probe heads 330. In other words, in an alternative construction, a plurality of guide channels similar to channel 352 may be provided at circumferential positions or other positions within balloon 351, and serve to accurately guide probe heads 330 in a desired direction.

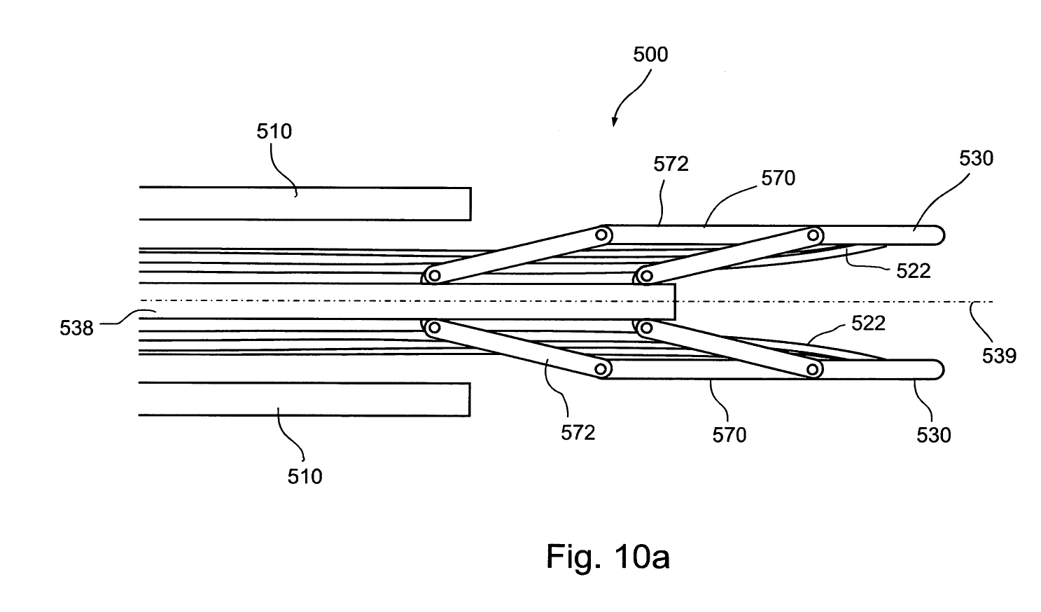

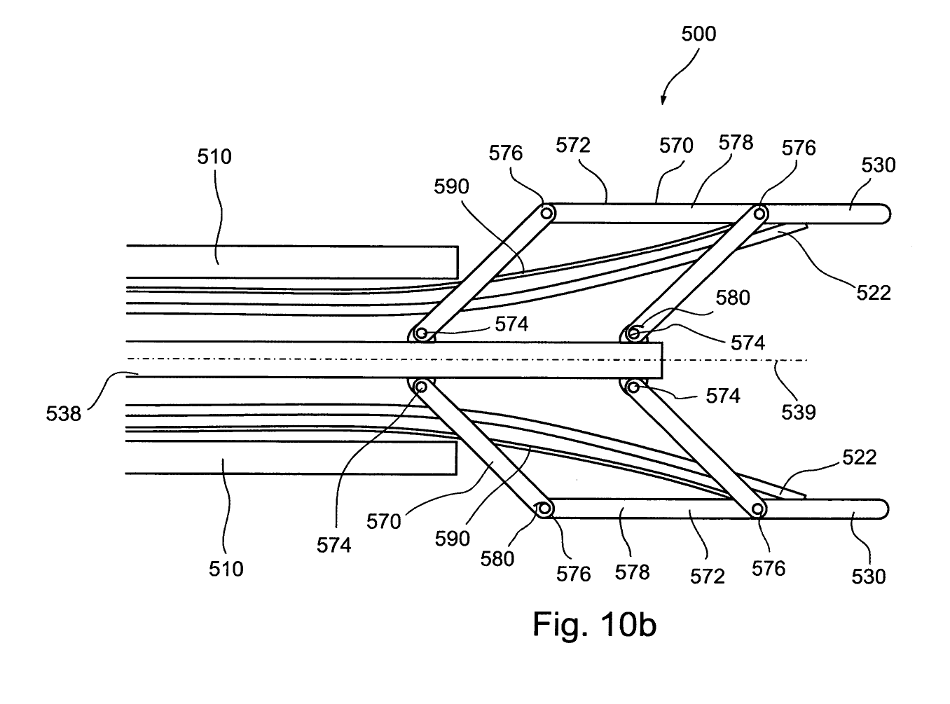

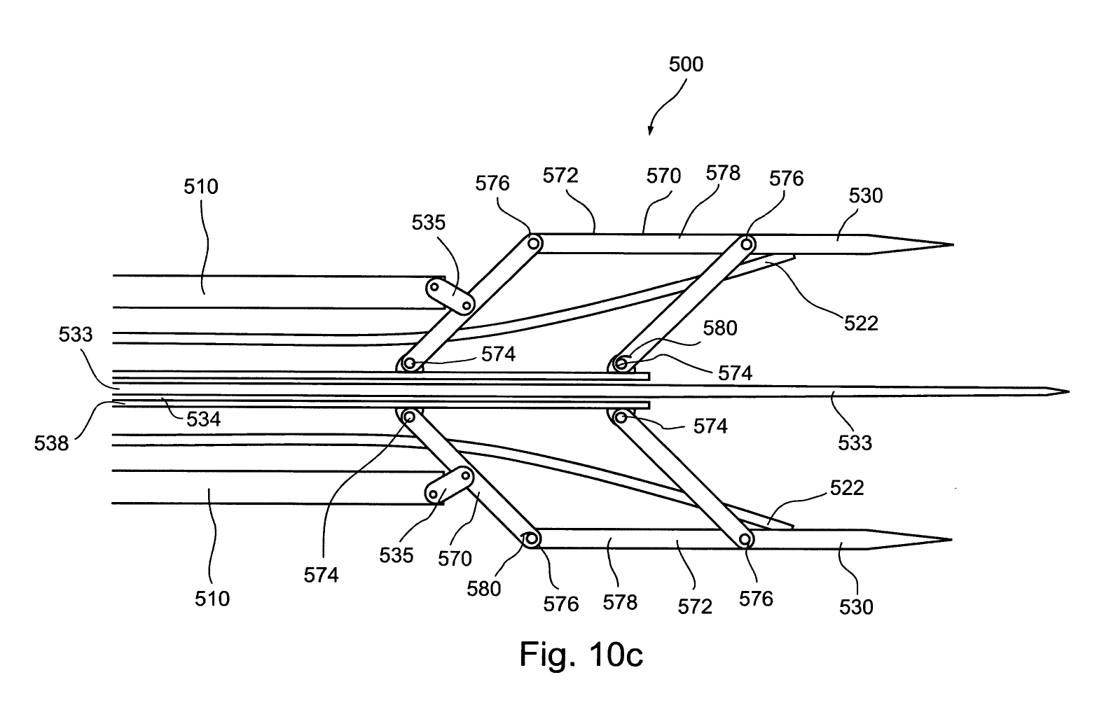

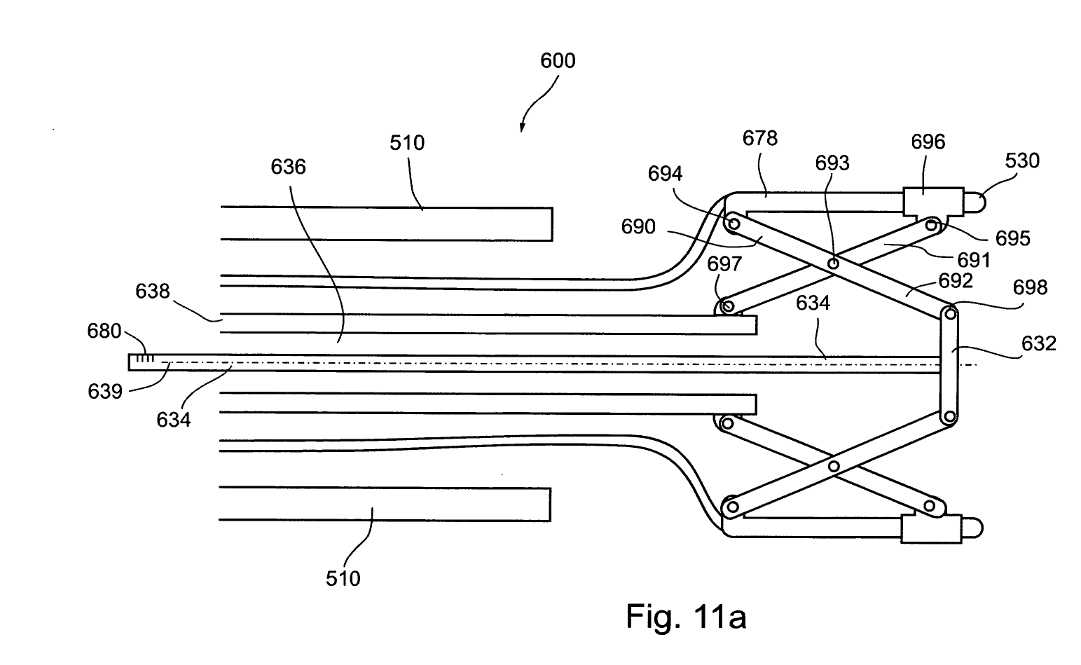

Attention is now draw to FIGS. 10a and 10b, which are simplified schematics of compact and extended configurations respectively of an introducer/probe combination utilizing a mechanical linkage for parallel extension of cryoprobes, according to an embodiment of the present invention.

FIGS. 10a and 10b present an apparatus 500 which may be used in a manner similar to that described for apparatus 200 discussed hereinabove. Apparatus 500 differs from apparatus 200 in that apparatus 500 further comprises a mechanical linkage 550 serving alternatively to collapse apparatus 500 into a compact configuration useful during insertion of apparatus 500 into a body, as shown in FIG. 10a, and to expand apparatus 500 into an extended configuration useful for cryo-treatment of a large ablation target, as shown in FIG. 10b. In an exemplary embodiment most clearly visible in FIG. 10b, cryoprobe heads 530 (two are shown in this exemplary FIG.) are linked to a cryogen supply source (not shown) by cryogen supply tubes 522 which are to be understood to contain a cryogen supply conduit 523 and cryogen exhaust conduit 524, constructed, for example, according to methods well known in the art. Each probe head 530 is supported by a mechanical linkage 570 operable to approach probe head 530 to a central support 538 and/or a central axis 539, and alternatively to distance head 530 therefrom, under control of a surgeon or other operator. In a preferred embodiment presented in FIGS. 10, mechanical linkage 570 is a parallelogram arrangement 572 comprising pivotal connections 574 to central support 538 and additional pivotal connections 576 to an arm 578 holding or comprising treatment heads 530. Optionally, treatment heads 530 may have sharpened distal ends to facilitate tissue penetration. A spring 580 may optionally be provided, optionally within one or more pivotal connections 574 and/or 576 or elsewhere. Spring(s) 580 may serve to cause arm(s) 578 to expand away from central support 538 or alternatively may serve to cause arms 578 to draw close to central support 538.

As may be seen in FIG. 10a, when arms 578 are drawn in close to central support 538, the entire mechanical linkage arrangement 570 and treatment heads 530 are operable to fit within an introducer portion 510 having characteristics similar to those described hereinabove with respect to introducers 210 and 310. When so configured, distal portions of apparatus 500 can easily be inserted (e.g. endoscopically, or through a trocar) into a body and caused to approach an ablation target.

When distal portions of apparatus 500 are positioned within a body cavity and near an ablation target, linkage 570 may be extended beyond a distal end of introducer 510, and arms 578 may be caused to expand away from central portion 538. The parallelogram structure of linkage 570/572 ensures that arms 578 and treatment heads 530 will be parallel to each other and parallel to central axis 539.

If springs 580 are configured to cause arms 578 to expand away from central portion 538, the distance by which central portion 538 is advanced within introducer 510 can provide means for controlled partial expansion of linkage 570. Alternatively, a mechanical stop may be provided to enable arms 578 to expand up to, but not beyond, a pre-determined distance, which pre-determined distance is preferably selected according to the principles presented hereinabove with respect to FIGS. 5, 6b and 7b.

In an alternative configuration, optional spring(s) 580 may be provided to cause linkage 570/572 to tend to contract into the position presented in FIG. 10a, with arms 578 close to central portion 538. In this case, optional pulling device 590, which may be a cord or wire, is provided to enable an operator, after extending central portion 538 from introducer 510, to pull arms 578 backwards towards introducer 510, thereby causing expansion of linkage 570/572 for purposes of deployment of treatment heads 530 in a expanded configuration.

Attention is now drawn to FIG. 10c, which is a simplified schematic similar to FIGS. 10a and 10b, showing additional alternative methods for control of deployment, according to an embodiment of the present invention.

FIG. 10c presents and embodiment of apparatus 500 wherein a mechanical link 535 comprising a pivot at either end links a proximal arm of mechanical linkage 570 with a distal end of introducer 510. As may be appreciated from the FIG., advancing central portion 538 beyond introducer 510 results in expansion of mechanical linkage 570 and expanded deployment of probe heads 530.

Attention is also drawn to an additional optional feature of apparatus 500 presented in FIG. 10c : distal ends of treatment heads 530 are here shown to be sharpened to facilitate penetration of heads 530 into target tissue. Sharp ends of treatment heads 530 may have conic points, simple or multi-faced chisel shapes, or be cut obliquely (in similarity to the form in which hypodermic needles are typically shaped) to facilitate penetration of heads 530 into tissue.

Attention is also drawn to an additional optional feature of apparatus 500. In FIG. 10c a centrally positioned probe 533 is provided, in characteristics and use similar to straight central probe 230b presented in FIGS. 2, 3, and 4, and to straight central probe 302 shown in FIGS. 9a and 9b, which straight probes are discussed in detail hereinabove. Probe 533 is positioned in a central lumen 534 within central portion 538, and may be advanced and retracted therein. In similarity to probes 230b and 302, probe 533 can be a therapeutic probe such as a cryoprobe, or can simply be an anchor probe used to anchor apparatus 500 to a target tissue prior to deployment and/or insertion of probe heads 530. In either case, probe 533 can be used to anchor apparatus 500 to target tissue to facilitate accurate deployment and/or insertion of probe heads 530, as described with respect to probe 230b hereinabove.

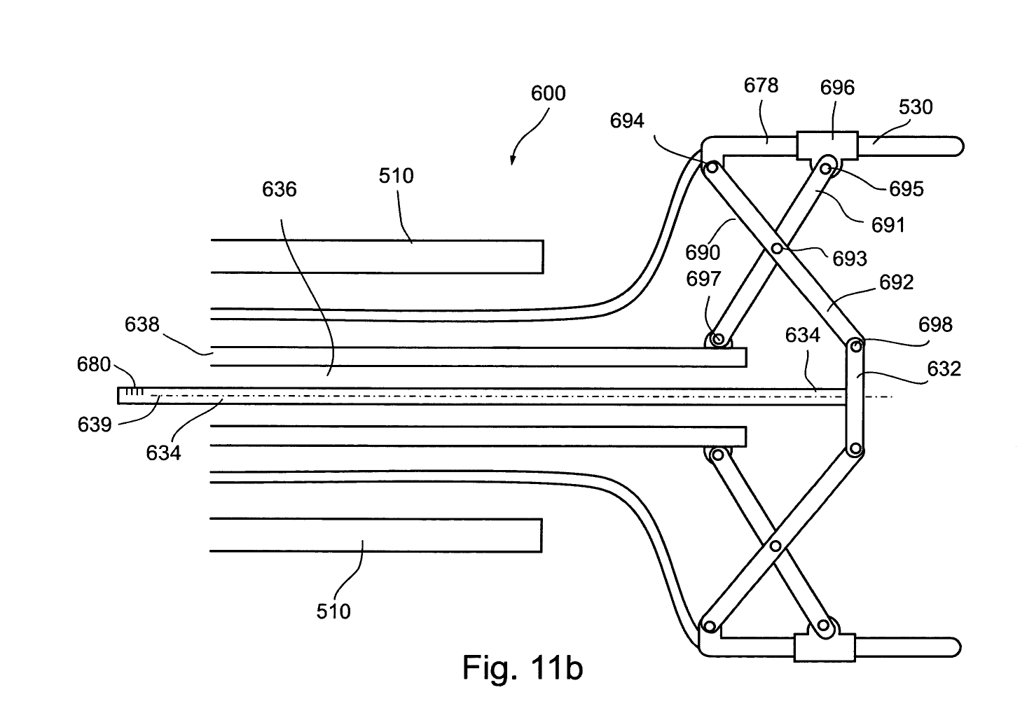

Attention is now drawn to FIGS. 11a and 11b, which are simplified schematics of compact and extended configurations respectively of an additional configuration of an introducer/probe combination utilizing a mechanical linkage for parallel extension of cryoprobes, according to an embodiment of the present invention.

FIGS. 11a and 11b present an apparatus 600 which is similar in most respects to apparatus 500 described above. Apparatus 600 differs from apparatus 500 in that in apparatus 600 mechanical linkage 570 embodied as an X-shaped linkage 573, serving (as does linkage 572 in apparatus 500) alternatively to expand arms 578 away from a central axis 539 and to contract arms 578 towards central axis 539, while maintaining parallel orientation of a plurality of arms 578.

Mechanical linkage 573 comprises an X-construction 690 shaped by two crossed arms 691 and 692 joined by a central pivot 693. A first corner 694 of X-construction 690 is pivotally connected to a proximal end of an arm 678. A second corner 695 of X-construction 690 is pivotally connected to a sleeve 696 within which arm 678 is free to move forwards and backwards. A third corner 697 of X-construction 690 is pivotally connected to a hollow central portion 638. Central portion 638 comprises a lumen 636 within which a central rod 636 is free to move forward and back. Rod 634 comprises a rod head 632. A fourth corner 698 of X-construction 690 is pivotally connected to rod head 632.

As may be seen from inspection of FIGS. 11a and 11b, when rod 634 is advanced within central portion 638, pivot 698 is forced away from pivot 697, “flattening” X-construction 690 and causing arm 678, and with it treatment head 530, to approach rod 634 and central axis 639 of apparatus 600. When rod 634 is retracted within central portion 638, pivot 698 is pulled towards pivot 697, causing a “heightening” of X-construction 690 and thereby causing arm 678 to be distanced from rod 634 and central axis 639. Since arms 678 are free to slide within sleeve 696 and arms 690 and 691 are (preferably) constructed to be of equal length, “heightening” and “flattening” of X-construction 690 causes arms 678 to move respectfully away from and towards rod 634 and axis 639, while maintaining arms 678 and treatment heads 530 in orientations parallel to that of axis 639. Distances of arms 678 from axis 639 may thus be precisely controlled by an operator. Optionally, a distal portion of rod 634 may comprise markings 680 visible to an operator during use of apparatus 600, which markings may be calibrated to show distances of arms 678 from axis 639 as a function of the forward-and-back position of rod 634 within central portion 638.

It is noted that system 600 may also comprise a centrally located anchoring probe (not shown in FIGS. 11a and 11b) similar to probe 533 of FIG. 10c. Such a probe might be positioned next to central rod 634, or rod 634 might be of hollow construction, leaving place for a central probe similar to probe 533 to be positioned within it, much as probe 533 is positioned within central support 538.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A probe deployment apparatus comprising an introducer and a plurality of probes each having a probe head, said apparatus providing
(a) a compact configuration wherein said probe heads are contained within said introducer; and
(b) a dispersed configuration wherein said probe heads extend from said introduce to form a distributed configuration wider than a diameter of said introducer, wherein distal tips of said probe heads are substantially parallel one to another.

2. The apparatus of claim 1, wherein, in said dispersed configuration, said distal tips are substantially parallel to a longitudinal axis of said introducer.

3. The apparatus of claim 1, wherein at least one of said probes comprises a cryocooler.

4. The apparatus of claim 1, wherein said probe heads assume a compact configuration when constrained by said introducer, and assume a dispersed configuration when unconstrained.

5. The apparatus of claim 1 wherein said probe heads are operable to be advanced and retracted within said introducer.

6. The apparatus of claim 5, wherein said probe heads are constrained to advance and retract in unison.

7. The apparatus of claim 5, wherein at least one of said probe heads may be advanced and retracted independently of another of said probe heads.

8. The apparatus of claim 1, wherein at least one of said probe heads comprises shape memory metal.

9. The apparatus of claim 8, wherein said at least one probe head comprises a cryocooler, and further comprises a portion which comprises shape memory metal which assumes an S-shaped configuration at a first temperature and another configuration at a second temperature.

10. The apparatus of claim 1, wherein at least one of said probe heads is pre-bent and assumes an S-shaped curve when unconstrained.

11. The apparatus of claim 10, wherein said plurality of probe heads extend from a common proximal shaft.

12. The apparatus of claim 11, wherein at least two of said probe heads comprise cryocoolers and said common shaft comprises a common cryogen exhaust lumen communicating with said cryocoolers.

13. The apparatus of claim 11, wherein at least two of said probe heads comprise cryocoolers and said common shaft contains a plurality of cryogen exhaust lumens each communicating with one of said cryocoolers.

14. The apparatus of claim 10, comprising a plurality of independently moveable probes.

15. The apparatus of claim 10, wherein a plurality of said probes are cryoprobes each having a cryogen input lumen and a cryogen exhaust lumen.

16. The apparatus of claim 10, wherein at leas some of said plurality of probes are constrained to advance and retract together.

17. The apparatus of claim 1, wherein a plurality of said probe heads comprise cryoprobe treatment tips coolable to cryoablation temperatures.

18. The apparatus of claim 17, wherein said treatment tips when in said dispersed configuration are configured to create a continuous cryoablation volume.

19. The apparatus of claim 17 wherein, while in said dispersed configuration, a first of said treatment tips is operable to create a cryoablation volume of radius D1 when operated in isolation, a second treatment tip, closest among said plurality of treatment tips to said first treatment tip, is operable to create a cryoablation volume of radius D2 when operated in isolation, and a distance D3 between said first treatment tip and said second treatment tip is greater than D1+D2.

20. The apparatus of claim 1, further comprising an inflatable balloon.

21. The apparatus of claim 20, wherein said inflatable balloon is operable to enforce separation of distal portions of said probe heads when said inflatable balloon is inflated.

22. The apparatus of claim 20, wherein said balloon comprises a channel sized to permit passage of a probe therethrough.

23. The apparatus of claim 22, wherein said balloon is annular in shape and said channel is positioned at a center of said annulus.

24. The apparatus of claim 20, wherein said balloon comprises a plurality of channels for guiding passage of a probe therethrough.

25. The apparatus of claim 24, wherein said channels are circumferentially positioned around said balloon.

26. The apparatus of claim 1, further comprising a mechanical linkage operable to modify distances of said distal tips of said probe heads from a longitudinal axis of said introducer.

27. The apparatus of claim 26, wherein said mechanical linkage comprises components formed as a parallelogram.

28. The apparatus of claim 27 further comprising a spring tending to maintain said mechanical linkage in compact position.

29. The apparatus of claim 27 further comprising a spring tending to maintain said mechanical linkage in expanded position.

30. The apparatus of claim 27 further comprising a pulling device which, when pulled, causes said mechanical linkage to assume an expanded configuration.

31. The apparatus of claim 26, wherein said mechanical linkage comprises a plurality of components formed as an X-shaped construction having a central pivot.

32. The apparatus of claim 26, wherein said mechanical linkage comprises a central rod which causes said linkage to expand laterally when pulled and causes said linkage to contract laterally when pushed.

33. A therapy apparatus which comprises a therapeutic probe having a base and a plurality of coolable probe heads extending from said base in substantially parallel directions.

34. The apparatus of claim 33, wherein a plurality of said probe heads comprise cryocoolers, and said base comprises a common cryogen exhaust lumen operable to exhaust cryogen from said cryocoolers.

35. The apparatus of claim 33, wherein a plurality of said probe heads comprise cryocoolers, and said base comprises a plurality of gas exhaust lumens each operable to exhaust cryogen from one of said cryocoolers.

36. The apparatus of claim 33, further comprising a sheath having a distal face which comprises a plurality of guide apertures, each guide aperture sized to accommodate one of said probe heads, at least some of said probe heads being operable to advance through one of said guiding apertures when said base is advanced within said sheath.

37. The apparatus of claim 36, wherein said sheath comprises thermally insulating material.

38. A method for delivering therapeutic probes to a therapeutic target, comprising:
(a) inserting into a body cavity an introducer containing a plurality of probes each having a probe head, said probe heads being contained in said introducer in a compact configuration;
(b) advancing said probe heads beyond said introducer, thereby causing said probe heads to assume a dispersed configuration wherein distal tips of said probe heads are substantially parallel one to another; and
(c) advancing said dispersed probe heads towards and into a treatment target.
